(19) United States
(12) Patent Application Publication
(10) Pub. No.: US 2024/0390447 A1
(43) Pub. Date: Nov. 28, 2024

(54) METHODS FOR SUPPLEMENTING IRON, PROMOTING IRON ABSORPTION, AND/OR IMPROVING SKIN CONDITION BY USING SASKATOON BERRY EXTRACT

(71) Applicant: TCI CO., LTD., Taipei (TW)

(72) Inventors: YUNG-HSIANG LIN, Taipei (TW); PEI-HAN CHANG CHIEN, Taipei (TW)

(21) Appl. No.: 18/430,680

(22) Filed: Feb. 2, 2024

Related U.S. Application Data
(60) Provisional application No. 63/503,503, filed on May 22, 2023.

(30) Foreign Application Priority Data
Dec. 29, 2023 (TW) 112151734

(51) Int. Cl. A61K 36/73; A61P 3/02; A61P 17/00

(52) U.S. Cl. CPC A61K 36/73; A61P 3/02; A61P 17/00; A61K 2236/331

(57) ABSTRACT

Methods for supplementing iron, promoting iron absorption, and/or improving skin condition are provided. The methods include administering to a subject in need thereof a composition that includes a Saskatoon berry extract. The Saskatoon berry extract is obtained by extracting Saskatoon berry (Amelanchier alnifolia) with water. The effect of improving skin condition includes reducing skin spots, reducing skin redness, smoothening skin texture, and improving coarse pores.

### CROSS-REFERENCES TO RELATED APPLICATIONS

This application claims the benefit of U.S. provisional application Ser. No. 63/503,503, filed on May 22, 2023 and claims the priority of Patent Application No. 112151734 filed in Taiwan, R.O.C. on Dec. 29, 2023. The entirety of the above-mentioned patent applications are hereby incorporated by references herein and made a part of the specification.

### REFERENCE OF AN ELECTRONIC SEQUENCE LISTING

The contents of the electronic sequence listing (P234552USI.xml; Size: 10,054 bytes; and Date of Creation: Jan. 25, 2024) is herein incorporated by reference in its entirety.

### BACKGROUND

### Technical Field

The present disclosure relates to a Saskatoon berry extract, and in particular to a Saskatoon berry extract with effects of supplementing iron, promoting iron absorption and/or improving skin condition.

### Related Art

With the changes of the times, people pursue a perfect appearance and a healthy physique. From the external appearance, skin quality, vital energy and blood to the internal collagen and iron content, or the nutritional supplementation of the body, there are very high requirements. Healthy skin and its complexion are a major factor in maintaining a radiant appearance. Therefore, people are increasingly paying attention to physical health and skin health care to maintain the best condition from inside out.

Iron is an essential nutrient for the human body, and its main function in the body is to produce heme. Generally, iron intake in daily diet can meet the needs of healthy men and postmenopausal women, but it cannot meet the larger needs of menstruating women, pregnant women and teenagers aged 15 to 49 and babies.

Studies have found that it is difficult for people with iron deficiency to meet their daily needs through diet. Iron in common foods mainly includes heme iron and non-heme iron. Although the heme iron is more easily absorbed, there remains a doubt over excessive absorption of trimethylamine oxide (TMAO; an important biological indicator of a cardiovascular disease risk), while the absorption rate of the non-heme iron is lower. Therefore, how to obtain sufficient iron intake in daily life has become the most common nutritional deficiency problem in the world. According to statistics from the World Health Organization, iron deficiency is not only prevalent in developing countries, but also remains a public health problem in developed countries. Moreover, there are many limitations on iron absorption in human intestinal tracts. Studies have found that 80% of the iron ingested by the human body is oxidized by a digestive tract into ferric ions (Fe³⁺) that are not easily absorbed, resulting in a loss of 65%-95% of the iron ingested in the intestinal tract.

### SUMMARY

Based on this, those skilled in the art urgently need to develop a functional food that can solve the above problems to benefit a vast number of people in need thereof.

In some embodiments, use of Saskatoon berry extract is provided, wherein the Saskatoon berry extract is used for preparation of a composition for supplementing iron or promoting iron absorption. The Saskatoon berry extract is obtained by extracting Saskatoon berry (Amelanchier alnifolia) with water.

In some embodiments, a method for supplementing iron or promoting iron absorption is provided, including administering to a subject in need thereof a composition including a Saskatoon berry extract. The Saskatoon berry extract is obtained by extracting Saskatoon berry (Amelanchier alnifolia) with water.

In some embodiments, the aforementioned promoting iron absorption is to promote iron absorption in an intestinal tract of the subject. In some embodiments, the Saskatoon berry extract promotes iron absorption in an intestinal tract of the subject to achieve the effect of promoting iron absorption.

In some embodiments, the aforementioned supplementing iron is to increase content of iron and ferritin in blood of the subject. In some embodiments, the Saskatoon berry extract increases content of iron and ferritin in the blood of the subject to achieve the effect of supplementing iron.

In some embodiments, use of Saskatoon berry extract is provided, wherein the Saskatoon berry extract is used for preparation of a composition for improving skin condition. The Saskatoon berry extract is obtained by extracting Saskatoon berry (Amelanchier alnifolia) with water.

In some embodiments, a method for improving skin condition is provided, including administering to a subject in need thereof a composition including a Saskatoon berry extract. The Saskatoon berry extract is obtained by extracting Saskatoon berry (Amelanchier alnifolia) with water.

In some embodiments, the aforementioned improving skin condition is to increase content of collagen in skin, increase secretion of elastin in the skin, or a combination thereof. In some embodiments, the Saskatoon berry extract increases content of collagen in skin, increases secretion of elastin in the skin, or a combination thereof to achieve the effect of improving skin condition.

In some embodiments, the aforementioned improving skin condition is to enhance expression of skin anti-aging genes in skin of the subject, and the aforementioned anti-aging genes are CCT2 gene, CCT6A gene, NADSYN gene, SOD3 gene, SIRT1 gene, or any combination thereof. In some embodiments, the Saskatoon berry extract enhances expression of skin anti-aging genes (such as CCT2 gene, CCT6A gene, NADSYN gene, SOD3 gene, SIRT1 gene, or any combination thereof) in skin of the subject to achieve the effect of improving skin condition.

In some embodiments, the aforementioned improving skin condition is to reduce skin spots, to reduce skin redness, to smoothen skin texture, to reduce coarse pores, or any combination thereof of the subject. In some embodiments, the Saskatoon berry extract reduces skin spots, reduces skin redness, smoothens skin texture, and reduces coarse pores, or any combination thereof to achieve the effect of improving skin condition.

In some embodiments, a weight ratio of the aforementioned water to Saskatoon berry for preparation of the Saskatoon berry extract is 10-20:1.

In some embodiments, the Saskatoon berry is extracted at 70° C. to 90° C.

In some embodiments, the Saskatoon berry is extracted for 50 min to 70 min.

In some embodiments, the aforementioned Saskatoon berry extract is obtained by extracting the Saskatoon berry with water at 85° C.±5° C. for 60 min, and a weight ratio of the water to the Saskatoon berry is 12:1.

In summary, the Saskatoon berry extract of the embodiments of the present disclosure is prepared by extracting the Saskatoon berry with the water, and can be used for preparation of the composition of supplementing iron, promoting iron absorption, and/or improving skin condition. In some embodiments, the aforementioned Saskatoon berry extract promotes iron absorption in the intestinal tract of the subject. In some embodiments, the aforementioned Saskatoon berry extract increases content of iron and ferritin in blood of the subject to achieve the effect of supplementing iron. In some embodiments, the aforementioned Saskatoon berry extract increases content of collagen, increases secretion of elastin, and enhances expression of anti-aging genes (such as CCT2 gene, CCT6A gene, NADSYN gene, SOD3 gene, SIRT1 gene, or any combination thereof) in skin of the subject to achieve the effect of improving skin condition. In some embodiments, the aforementioned Saskatoon berry extract reduces skin spots, reduces skin redness, smoothens skin texture, reduces coarse pores, or any combination thereof to achieve the effect of improving skin condition.

### BRIEF DESCRIPTION OF THE DRAWINGS

FIG. 1 is a bar chart showing experimental results of relative content of human ferritin;

FIG. 2 is a bar chart showing experimental results of relative content of collagen;

FIG. 3 is a bar chart showing experimental results of relative secretion of elastin;

FIG. 4 is a bar chart showing experimental results of relative expression level of anti-aging genes;

FIG. 5 is a bar chart showing experimental results of relative expression level of SIRT1 gene;

FIG. 6 is a bar chart showing experimental results of content of iron in blood of the subjects at week 0 and week 4;

FIG. 7 is a bar chart showing experimental results of content of ferritin in blood of the subjects at week 0 and week 4;

FIG. 8 is a bar chart showing experimental results of relative percentage of skin spots in the subjects at week 0 and week 4;

FIG. 9 is photographs showing skin spots of a subject at week 0 and week 4;

FIG. 10 is a bar chart showing experimental results of relative percentage of skin redness of subjects at week 0 and week 4;

FIG. 11 is photographs showing skin redness and spots of a subject at week 0 and week 4;

FIG. 12 is a bar chart showing experimental results of relative percentage of skin texture of the subjects at week 0 and week 4;

FIG. 13 is photographs showing skin texture of a subject at week 0 and week 4;

FIG. 14 is a bar chart showing experimental results of relative percentage of pores of the subject at week 0 and week 4; and

FIG. 15 is photographs showing pores of a subject at week 0 and week 4.

### DETAILED DESCRIPTION

Herein, Excel software is used for statistical analysis. Data is represented by mean±standard deviation (SD), and differences between groups are analyzed with a student's t-test. In the figure, “*” or “#” represents that p value is less than 0.05, “**” or “##” represents that p value is less than 0.01, and “***” or “###” represents that p value is less than 0.001. The more the “*” or “#”, the more significant the statistical differences.

The term “extract” refers to a product prepared by extraction. The extract can be presented in the form of a solution dissolved in a solvent, or the extract can be presented as a concentrate or essence without or substantially without a solvent, or can be presented as dried powder.

Saskatoon berry is fruits of Amelanchier alnifolia. The Saskatoon berry is a unique blueberry variety in Canada. It is produced in the “Grain Basket”—Saskatchewan, the province in Canada with the longest sunshine and fertile land with rich minerals. The Saskatoon berry has abundant nutrients and about 3.5 folds higher content of iron than blueberries. In some embodiments, the Saskatoon berry refer to whole fruits, fruits with peel, peeled fruits, fruit containing seeds, or fruit with seeds removed. By way of examples, the whole fruit of the Saskatoon berry includes peel, flesh, and seeds.

In some embodiments, after whole fruits of Saskatoon berry (Amelanchier alnifolia) are broken up with a breaker, the broken up whole fruits are mixed with an extraction solvent (e.g., water) in a specific weight ratio, and then, the mixture is extracted at a specific temperature to obtain a mixed solution containing solids. Next, an initial liquid extract is filtered to remove fine solid impurities. Next, the filtered mixed solution is concentrated to obtain a concentrated solution. Next, the concentrated solution is sterilized to obtain a sterilized liquid Saskatoon berry extract. The concentrated solution is dried into powder by spray drying to obtain a solid Saskatoon berry extract. By way of examples, the extraction solvent may be the water; the specific weight ratio may be 10-20:1; the specific temperature may be 70° C. to 90° C.; and specific time may be 50 min to 70 min.

Here, a specific proportion of an extraction solvent and a substance to be extracted (such as the crushed fruits) or specific extraction time can significantly improve the extraction efficiency; and the specific extraction time can avoid the possible degradation of active ingredients in the extract due to overlong extraction time.

In some embodiments, the Saskatoon berry extract is prepared by breaking up whole fruits of Saskatoon berry and then mixing the crushed whole fruits with water in a weight ratio of 10-20:1, and extracting the mixture at 70° C. to 90° C. for 50 min to 70 min. By way of examples, the Saskatoon berry extract is prepared by mixing fruit powder of the Saskatoon berry broken up with the water in a weight ratio of 1:12, and extracting the mixture at 85° C.±5° C. for about 60 min.

In some embodiments, the mixed solution obtained by extraction is filtered through a 400-mesh filter to remove fine solids, and the filtered mixed solution is concentrated under reduced pressure to obtain the Saskatoon berry extract. By way of examples, the temperature of the concentration under reduced pressure may be 60° C.±5° C.

In some embodiments, the filtered mixed solution is concentrated under reduced pressure until an established specification is met to obtain the Saskatoon berry extract, and this established specification is to stop concentration when degrees Brix of the filtered mixed solution are 8.5±0.5 to obtain the Saskatoon berry extract.

In some embodiments, if a pH value of the aforementioned concentrated Saskatoon berry extract is higher than 3.7, 0.1% of malic acid can be added to make its pH value 2.7±1.0.

In some embodiments, the Saskatoon berry extract has functions of iron supplementation and iron absorption promotion. By way of examples, the iron supplementation is to increase content of iron and ferritin in blood, while the iron absorption promotion is to promote iron absorption in an intestinal tract. The level of iron in the body is maintained within an optimal physiological range by the iron absorption in the intestinal tract, and when the body lacks the iron, anemia is caused easily to cause paleness. Therefore, when a subject takes the Saskatoon berry extract, the iron absorption of his/her intestinal tract can be effectively promoted, and his/her skin is kept ruddy.

In some embodiments, the Saskatoon berry extract improves skin condition. By way of examples, the aforementioned improving skin condition is to increase content of collagen, increase secretion of elastin, or a combination thereof in skin of the subject. Specifically, the Saskatoon berry extract effectively promotes production of the collagen and the elastin in the skin of the subject, and keeps his/her skin elastic, smoothens fine lines, and maintains gloss.

By way of examples, the aforementioned skin improvement is to enhance expression of skin anti-aging genes, and the aforementioned anti-aging genes are chaperonin containing TCP1 subunit 2 (CCT2) gene, chaperonin containing TCP1 subunit 6A (CCT6A) gene, glutamine-dependent NAD⁺ synthetase (NADSYN) gene, superoxide dismutase 3 (SOD3) gene, NAD-dependent deacetylase (Sirtuin 1, SIRT1) gene, or any combination thereof. When the subject takes the Saskatoon berry extract, the expression of the aforementioned skin anti-aging genes can be effectively promoted, skin aging can be slowed down, and the skin is kept young.

By way of examples, the Saskatoon berry extract reduces skin spots, reduces skin redness, smoothens skin texture, and reduces coarse pores, or any combination thereof is realized. In other words, after the subject takes the Saskatoon berry extract, the skin spots can be reduced to fade color spots, the skin redness can be reduced to soothe red skin, and the skin texture can be reduced to smoothen rough texture, and/or coarse pores can be reduced.

In some embodiments, the aforementioned subject is a human.

In some embodiments, any of the aforementioned compositions may be a pharmaceutical product. In other words, the pharmaceutical product includes an effective content of Saskatoon berry extract.

In some embodiments, the aforementioned pharmaceutical product can be manufactured by using a technology known to those skilled in the art into a dosage form suitable for being enterally, parenterally, orally or topically administrated.

In some embodiments, the enterally or orally administrated dosage form may be, but is not limited to, a tablet, a troche, a lozenge, a pill, a capsule, dispersible powder or a granule, a solution, a suspension, an emulsion, syrup, an elixir, slurry, or the like. In some embodiments, the parenterally or topically administrated dosage form may be, but is not limited to, an injection, sterile powder, an external preparation, or the like. In some embodiments, an administration mode of the injection may be, but is not limited to, subcutaneous injection, intraepidermal injection, intradermal injection, or intralesional injection.

In some embodiments, the aforementioned pharmaceutical product may include a pharmaceutically acceptable carrier that is widely used in a drug manufacturing technology. In some embodiments, the pharmaceutically acceptable carrier may be one or more of the following carriers: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, and the like. The type and quantity regarding the carrier selected falls within the scope of the professional quality and routine technology known to those skilled in the art. In some embodiments, a solvent used as the pharmaceutically acceptable carrier may be water, normal saline, phosphate buffered saline (PBS), or an aqueous solution containing alcohol.

In some embodiments, any of the aforementioned compositions may be an edible product for non-medical use. In other words, the edible product includes a specific content of Saskatoon berry extract. In some embodiments, the edible product may be a general food, a health-care food, or a dietary supplement.

In some embodiments, the aforementioned edible product can be manufactured by using a technology well known to those skilled in the art into a dosage form suitable for oral administration. In some embodiments, the aforementioned general food may be an edible product itself. In some embodiments, the general food may be, but is not limited to, beverages, fermented foods, bakery products, or seasonings.

In some embodiments, the obtained Saskatoon berry extract can be further used as a food additive to prepare a food composition containing the Saskatoon berry extract. Here, it is possible to add the Saskatoon berry extract of any embodiment during preparation of raw materials by a well-known method, or to add the Saskatoon berry extract of any embodiment in the food production process to formulate, with any edible material, an edible product (i.e., food composition) for human and non-human animal consumption.

In some embodiments, the Saskatoon berry extract contained in the aforementioned composition containing the Saskatoon berry extract may be liquid or solid. By way of examples, the solid may be powder or a tablet.

In some embodiments, the usage amount of the composition is at least 1.2 g/day of liquid Saskatoon berry extract.

In some embodiments, the usage amount of the composition is at least 0.2 g/day of solid Saskatoon berry extract.

### Example 1: Preparation of Saskatoon Berry Extract

Firstly, whole fruits of Saskatoon berry (fruit of Amelanchier alnifolia) (origin: Canada) were taken and crushed with a breaker (brand: SAMPO 1.5 L KJ-SD15G). Next, water was mixed with whole fruit pieces of the Saskatoon berry in a weight ratio of 12:1, and the mixture was extracted at 85° C.±5° C. for 60 min to form a mixed solution containing solids. Next, the mixed solution containing the solids was filtered through a 400-mesh filter to remove fine solids from the mixed solution. Concentration was stopped when the filtered mixed solution was concentrated under reduced pressure by a concentrator (brand/model: BUCHI-Rotavapor R-100) at 60° C.±5° C. until degrees Brix of the solution were 8.5±0.5 to obtain a liquid Saskatoon berry extract. Here, if a pH value of the concentrated Saskatoon berry extract was higher than 3.7, 0.1% of malic acid was added to make its pH value 2.7±1.0.

### Example 2: Test of Iron Absorption in Intestinal Tract

Ferritin can serve as an indicator of iron content in the body. Here, measurement of the ferritin can serve as a judgment standard for determining iron absorption in intestinal cells.

Cells used were intestinal epithelial cells (hereinafter referred to as Caco-2 cells) (ATCC HBT-37™). A cell medium used was prepared from 80% of DMEM medium (Dulbecco's Modified Eagle Medium; brand: Gibco, Cat. 12100-038) supplemented with 20% of decomplementized fetal bovine serum (fetal bovine serum brand: Gibco, Cat. 10438-026; obtained by heating fetal bovine serum at 56° C. for 30 min), 1% of non-essential amino acid solution (NEAA; brand: Gibco, 11140035), 1% of L-glutamine (brand: Gibco, 25030081) and 1% of antibiotic-antimycotic (brand: Gibco, Cat. 15140-122). An iron-free medium used was a minimum essential medium (brand: Gibco) supplemented with 10 mmol/L of piperazine-1,4-diethanesulfonic acid (PIPES; brand: Thermo), 4 mg/L of hydrocortisone (brand: Thermo), and 5 μg/L of sodium selenite (brand: Thermo), 34 μg/L of triiodothyronine (brand: Thermo), 5 mg/L of insulin (brand: Thermo), 20 μg/L of epidermal growth factor (brand: Thermo), and 1% of antibiotic-antimycotic.

The Caco-2 cells were implanted at a density of 2.0×10⁴ cells per well into a 24-well culture plate containing 500 μL of cell medium per well, and were placed in an incubator with a 5% carbon dioxide concentration and a temperature of 37° C. and cultured for 14 days. Moreover, the cell medium was replaced every three days, and a test of iron absorption was conducted after 14 days of culture.

2 days prior to the test of iron absorption, the cell medium in the 24-well culture plate was removed, and after washing with 1×PBS (brand: Gibco), the cell medium was replaced with an iron-free medium for culture.

Groups were divided into three groups: a blank group, a control group and an experimental group. After two days of culture with the iron-free medium, the iron-free medium in the 24-well culture plate was replaced with an experimental medium. Here, the experimental medium in the experimental group was an iron-free medium containing 0.125 mg/mL of Saskatoon berry extract prepared in Example 1, the experimental medium in the control group was an iron-free medium containing 100 μm of ascorbic acid (Sigma), and the experimental medium in the blank group was an iron-free medium without addition of other ingredients. Then, 10 μmol/L of ferrous gluconate was added into each of the three groups to serve as an iron source, and culture was performed for 24 h in an incubator with a 5% carbon dioxide concentration and a temperature of 37° C.

Next, the Caco-2 cells in the three groups were collected with 200 μL of RIPA buffer (brand: Thermo) to obtain a cell solution, and the concentration of protein in the cell solution was measured by Bradford protein assay, and then, the content of human ferritin in the cell solution was measured with a human ferritin (FTL) ELISA kit (brand: Abcam). The content of the human ferritin measured in the blank group was considered 100%. It should be particularly noted that statistically significant differences between the groups were analyzed through a student's t-test, as shown in FIG. 1. Moreover, in FIG. 1, “**” represents that p values of the control group and the experimental groups are less than 0.05 in comparison with that in the blank group.

See FIG. 1. Compared with the blank group, the content of human ferritin in the control group was 107.50%, and the content of human ferritin in the experimental group was 108.70%. It could be known that the content of human ferritin in the experimental group was increased significantly, indicating that the Caco-2 cells treated with the Saskatoon berry extract for 24 h produced more human ferritin serving as an indicator of human iron storage. From this, it could be known that the Saskatoon berry extract had functions of promoting iron absorption by intestinal cells to produce ferritin.

Based on this, it could be known that the Saskatoon berry extract had the functions of promoting iron absorption in the intestinal cells to produce ferritin. After a subject took the Saskatoon berry extract, iron absorption in the intestinal cells of the subject was promoted and ferritin was produced, thereby increasing the content of iron in the body of the subject, and achieving the effect of iron supplementation, and thus, a ruddy complexion of skin is promoted and maintained.

### Example 3: Collagen Secretion Experiment

Here, a cell medium used (hereinafter referred to as an MEM medium) was formulated with 90% of minimum essential medium (brand; Gibco, Cat. 11095080), 10% of fetal bovine serum protein (FBS; brand: Gibco, Cat. 10437-028), 1% of antibiotic-antimycotic (brand: Gibco, Cat. 15240-062) and 1 mM sodium pyruvate (brand: Gibco, Cat. 11360-070). A cell line used was a CCD-966Sk cell (ATCC; Cat. CRL-1881).

Firstly, 2×10⁴ CCD-966Sk cells were taken and placed in a 24-well cell culture plate containing 0.5 mL of MEM medium per well, and cultured at 37° C. for 24 h. The CCD-966Sk cells were attached to the bottom of the cell culture plate, and then washed once with 1×DPBS (Dulbecco's phosphate-buffered saline, brand: Gibco, Cat. 14200-075).

The CCD-966Sk cells were divided into an experimental group and a blank group. The MEM medium in each group was removed and replaced with 0.5 mL experimental medium per well, then the experimental medium was placed at 37° C. and culture was further performed for 48 h. The experimental medium in the experimental group was an MEM medium containing 0.125 mg/mL of Saskatoon berry extract obtained in Example 1. The experimental medium in the blank group was a pure MEM medium (i.e., an MEM medium without the Saskatoon berry extract).

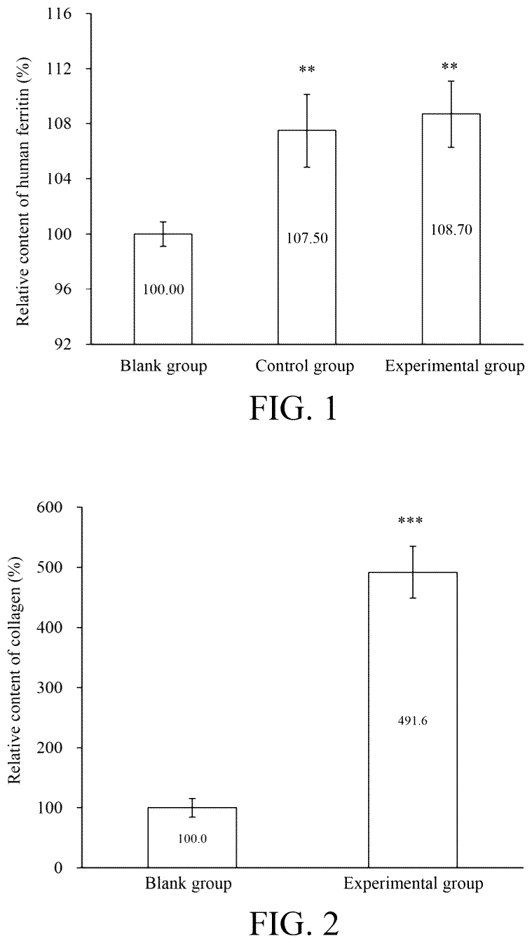

1 mL of experimental medium in each group was collected, and treated with a soluble collagen assay kit (brand: Biocolor, Cat. S1000) to form a solution to be detected in each group. Next, absorbance of solution to be detected in each group at 555 nm was measured using a spectrophotometer, and statistical analysis was performed using a student's t-test in Excel software, as shown in FIG. 2. Here, the p value symbol “***” in FIG. 2 was relative to that in the blank group.

See FIG. 2. Values in the blank group were treated as 100%. Compared with the blank group, the relative content of collagen in the experimental group was 491.6%, indicating that the Saskatoon berry extract effectively promotes the cells to secrete 4.9 folds of collagen. In other words, when a subject took the Saskatoon berry extract, his/her skin cells can effectively secrete collagen, thereby improving the skin of the subject, keeping the skin of the subject elastic, smoothening fine lines, and maintaining glossiness.

### Example 4: Elastin Experiment

Here, a cell medium used (hereinafter referred to as an MEM medium) was formulated with 90% of minimum essential medium (brand: Gibco, Cat. 11095080), 10% of fetal bovine serum protein (FBS; brand: Gibco, Cat. 10437-028), 1% of antibiotic-antimycotic (brand: Gibco, Cat. 15240-062) and 1 mM sodium pyruvate (brand: Gibco, Cat. 11360-070). A cell line used was a human skin fibroblast (CCD-966Sk cell, brand: ATCC®, CRL-1881), hereinafter referred to as a CCD-966Sk cell.

The CCD-966Sk cells were inoculated at a density of 1×10⁵ cells per well into a 6-well culture plate containing 2 ml MEM medium per well, and cultured at 37° C. for 24 h. Here, the cultured CCD-966Sk cells were divided into two groups: an experimental group and a blank group. Next, the MEM medium in each group was replaced with a corresponding experimental medium. Regarding the experimental group, culture was then performed at 37° C. for 48 h and secretion of elastin was detected after the culture. Regarding the blank group, the secretion of the elastin was detected immediately after the MEM medium was replaced with the experimental medium. The experimental medium in the experimental group was an MEM medium containing 0.0625 mg/mL of Saskatoon berry extract prepared in Example 1. The experimental medium in the blank group was a pure MEM medium (i.e., an MEM medium without the Saskatoon berry extract).

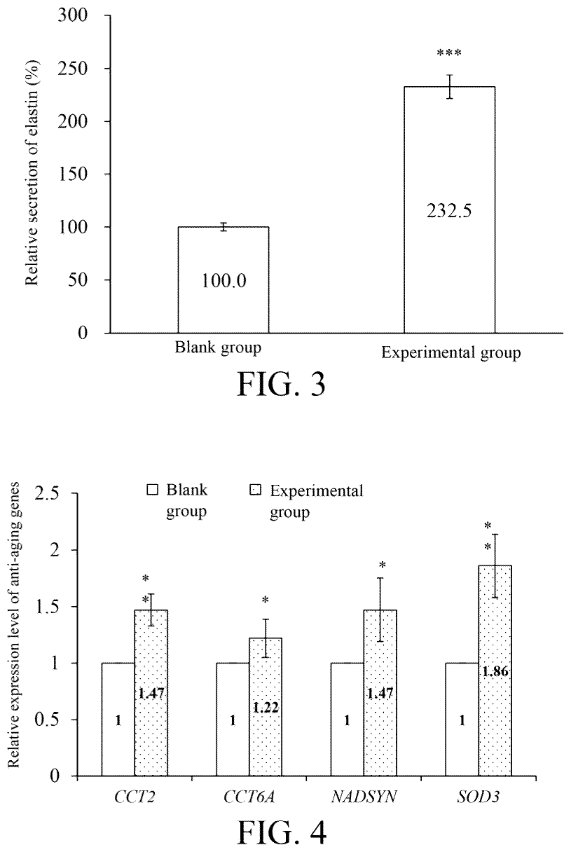

In the detection of the secretion of the elastin, after the CCD-966Sk cells in each group were treated with a Fastin™ elastin assay kit (brand: Biocolor), a wavelength was set as 513 nm with an ELISA reader (brand: BioTek) to detect the secretion of the elastin of the CCD-966Sk cells in the three groups, as shown in FIG. 3. The relative secretion of the elastin measured in the blank group where culture was not performed with the experimental medium for 24 h was considered 100%. Here, the p value symbol “***” in FIG. 3 was relative to that in the blank group.

See FIG. 3. Compared with the blank group, the relative secretion of elastin in the experimental group was 232.5%. In other words, the relative secretion of elastin in the experimental group was significantly increased by 2.3 folds in comparison with that in the blank group. From this, it could be known that the Saskatoon berry extract had a function of promoting synthesis of elastin by cells, such that the skin of a subject was kept elastic, fine lines were smoothened, and glossiness was maintained.

### Example 5: Skin Anti-Aging Gene Experiment

Here, skin anti-aging genes detected were CCT2 gene (Gene ID: 10576), CCT6A gene (Gene ID: 908), NADSYN gene (Gene ID: 6098), SOD3 gene (Gene ID: 6649), and SIRT1 gene (Gene ID: 23411). A cell medium used was a minimum essential medium (Eagle) (MEM (Eagle)) (brand: Gibco; Cat. 11095080) supplemented with 10% (v/v) fetal bovine serum (brand: Gibco; Cat. 10437-028). The minimum essential medium (brand: Eagle) was formulated by adding additional ingredients to an Earle's balance salt solution (Earle's BSS), where the additional ingredients include 1 mM sodium pyruvate, 1.5 g/L of sodium bicarbonate, and 0.1 mM of non-essential amino acid solution. A cell line used was a CCD-966Sk cell.

Firstly, 1.5×10⁵ CCD-966Sk cells were taken and placed in a 24-well cell culture plate containing 2 mL of cell medium per well, and cultured at 37° C. for 24 h. Next, the CCD-966Sk cells were divided into an experimental group and a blank group. Moreover, the CCD-966Sk cells of the experimental group were attached to the cell culture plate, and then a sample to be detected was added into the cell medium of the cell culture plate and treated at 37° C. for 24 h. Here, the sample to be detected in the experimental group was 0.125 mg/mL of Saskatoon berry extract prepared in Example 1.

Next, supernatant in each group was removed, and a cell lysate from an RNA extraction reagent kit (purchased from Geneaid, Taiwan, Lot No. FC24015-G) was added for a subsequent RNA extraction process.

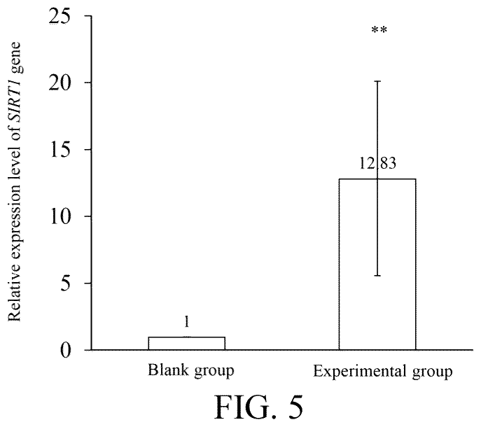

The CCD-966Sk cells in each group were collected, and RNA in each group was extracted with the RNA extraction reagent kit. Next, 1000 ng of RNA was taken from each group as a template, and the RNA was reversely transcribed into corresponding cDNA with SuperScript® III reverse transcriptase (purchased from Invitrogen, USA, Cat. 18080-051). Then a quantitative real-time reverse transcription polymerase chain reaction was carried out on the cDNA in each group by an ABI StepOnePlus™ real-time PCR system (Thermo Fisher Scientific, USA), KAPA SYBR FAST (purchased from Sigma, USA, Cat. 38220000000) and primers in Table 1 (SEQ ID NO: 1 to SEQ ID NO: 10) to observe an expression level of skin anti-aging genes in the CCD-966Sk cells. Instrument set conditions for the quantitative real-time reverse transcription polymerase chain reaction were as follows: a reaction was carried out at 95° C. for 20 s, followed by a reaction at 95° C. for 3 s, and a reaction at 60° C. for 30 s, which was repeated for 40 cycles, and gene quantification was performed using a 2-ΔCt method, as shown in FIGS. 4 and 5. Here, the quantitative real-time reverse transcription polymerase chain reaction by the cDNA can indirectly quantify an mRNA expression level of a gene, thereby inferring an expression level of a protein coded by the gene.

TABLE 1

| Target gene | Primer name | Sequence number | Primer sequence |
|---|---|---|---|
| CCT2 | CCT2-F | SEQ ID NO: 1 | AAGCCACGAAGGCTGCAA |
| | CCT2-R | SEQ ID NO: 2 | TCATCGGAACCATGATCAACTG |
| CCT6A | CCT6A-F | SEQ ID NO: 3 | TGGCCAGAACATCTCTTCGTACT |
| | CCT6A-R | SEQ ID NO: 4 | AGTCCACTACAGCCTCTGTTAAGACA |
| NADSYN | NSDSYN-F | SEQ ID NO: 5 | GCAAAATGTGCAGGCTCGAA |
| | NSDSYN-R | SEQ ID NO: 6 | GCACTGGAGCAGTCGTACTT |
| SOD3 | SOD3-F | SEQ ID NO: 7 | AGCTGGAAAGGTGCCCGA |
| | SOD3-R | SEQ ID NO: 8 | CTTGGCGTACATGTCTCGGAT |
| SIRT1 | SIRT1-F | SEQ ID NO: 9 | TAGCCTTGTCAGATAAGGAAGGA |
| | SIRT1-R | SEQ ID NO: 10 | ACAGCTTCTTCACAGTCAACTTTGT |

In Table 1, F is a forward primer and R is a reverse primer.

See FIG. 4. Relative expression levels of the CCT2 gene, the CCT6A gene, the NADSYN gene, and the SOD3 gene in the blank group were considered as 1, while compared with that in the blank group, relative expression levels of the CCT2 gene, the CCT6A gene, the NADSYN gene, and the SOD3 gene in the experimental group were 1.47, 1.22, 1.47, and 1.86, respectively, indicating that the expression level of each skin anti-aging gene expressed by the CCD-966Sk cells treated with the Saskatoon berry extract was increased. Therefore, when a subject took the Saskatoon berry extract, the expression levels of the CCT2 gene, the CCT6A gene, the NADSYN gene, and the SOD3 gene were increased, thereby delaying skin aging of the subject and maintaining a youthful state.

See FIG. 5. A relative expression level of the SIRT1 gene in the blank group was considered as 1, while compared with that in the blank group, a relative expression level of the SIRT1 gene in the experimental group was 12.83, indicating that the expression level of the SIRT1 gene expressed by the CCD-966Sk cells treated with the Saskatoon berry extract was increased by 12.8 folds. Therefore, when a subject took the Saskatoon berry extract, the expression level of the SIRT1 gene was increased, thereby delaying skin aging of the subject and maintaining a youthful state.

### Example 6: Human Experiment

Here, human experiments were conducted in a self-control manner, and blood and skin condition changes of 10 subjects before and after taking a drink containing the Saskatoon berry extract were compared. The drink used contained 1.2 g of Saskatoon berry extract obtained in Example 1 per bottle.

Test mode: The 10 subjects took a bottle of drink containing 1.2 g of Saskatoon berry extract daily for 4 consecutive weeks, and blood collection and skin detection were conducted before taking (week 0) and 4 weeks after taking (week 4). The 10 subjects were over 20 years old and were known to have iron deficiency. Here, the drink was composed of 1.2 g of Saskatoon berry extract and 48.8 mL of water, with a total weight of 50 mg.

Blood detection: 6 mL of venous blood was collected from the subjects using EDTA anticoagulant-containing purple headed blood collection tubes, and blood detection was performed (entrusted to Lezen Reference Lab). Detection items include detection of content of iron (Fe) and ferritin in blood of the subjects. Generally, a reference value for the content of the iron in the blood was 50 μg/dL to 122 μg/dL, while a reference value for the content of the ferritin in the blood was 11 μg/dL to 306.8 μg/dL.

Skin detection: Facial skin was photographed using an RBX polarized light technology of a VISIA complexion analysis system (Canfield scientific, USA) to detect skin spots, textures, pores, and redness.

It should be particularly noted that statistical analysis was performed through a student's t-test on statistically significant differences between measurement results from week 0 and week 4. “*” represents a p value compared with that at week 0.

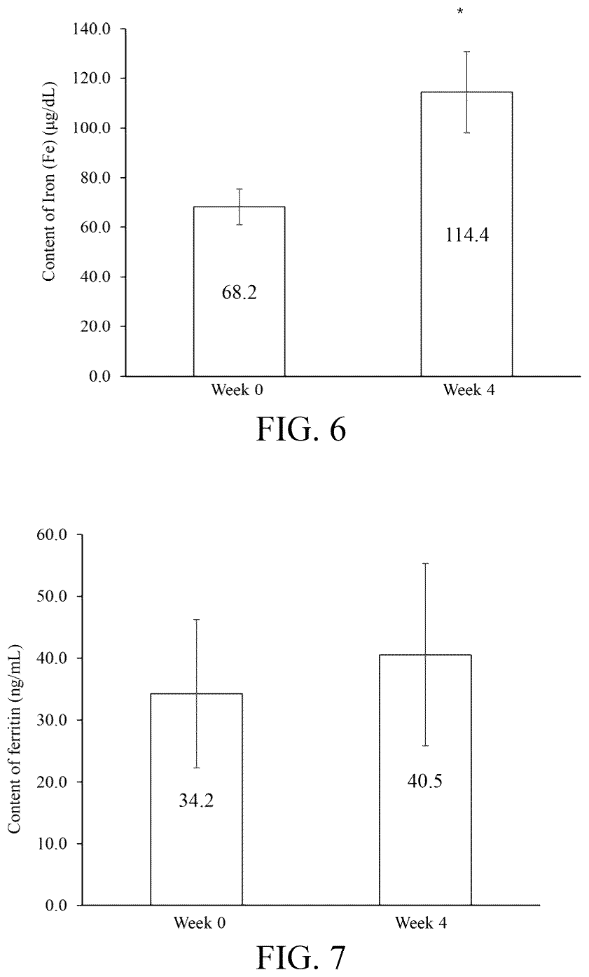

See FIG. 6. Average content of iron in the blood of the 10 subjects detected at week 0 was 68.2 μg/dL, and after taking the drink containing the Saskatoon berry extract for 4 consecutive weeks, the 10 subjects had their average content of iron in the blood detected at week 4 increased to 114.4 μg/dL. That is, the average content of iron in the blood of the 10 subjects was increased by 67.7%. Moreover, 9 out of the 10 subjects felt improvement in their content of iron, indicating that the percentage of improved individuals was 90%. From this, it could be known that taking a composition containing the Saskatoon berry extract effectively help a subject supplement iron and increased the content of iron in the blood of the subject.

See FIG. 7. Average content of ferritin in the blood of the 10 subjects detected at week 0 was 34.2 μg/dL, and after taking the drink containing the Saskatoon berry extract for 4 consecutive weeks, the 10 subjects had their average content of ferritin in the blood detected at week 4 increased to 40.5 μg/dL. That is, the 10 subjects had their average content of ferritin in the blood increased by 18.5%. Moreover, 6 out of the 10 subjects felt improvement in their content of ferritin, indicating that the percentage of improved individuals was 60%. From this, it could be known that taking the composition containing the Saskatoon berry extract effectively help a subject supplement iron and increased the content of ferritin in the blood of the subject.

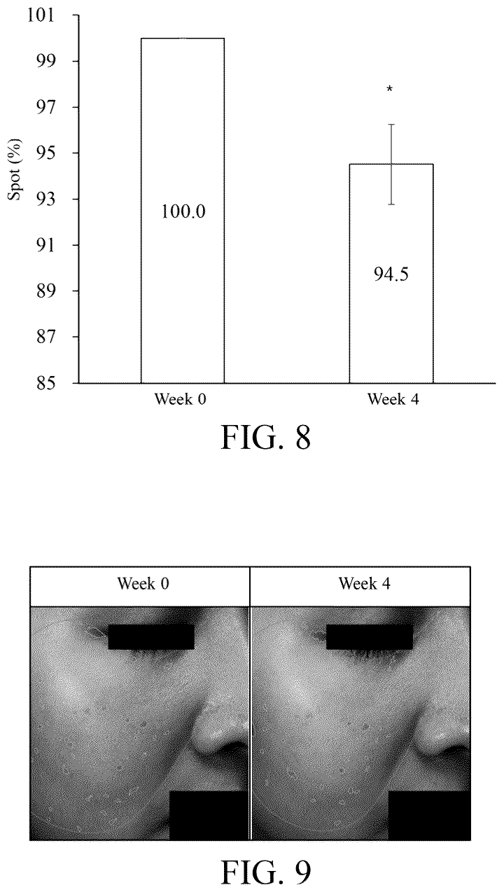

See FIG. 8. A mean of skin spots of the 10 subjects measured at week 0 was considered as 100%. Compared with week 0, a mean of skin spots measured at week 4 was considered as 94.5%. In other words, after taking the drink containing the Saskatoon berry extract for 4 consecutive weeks, the 10 subjects had a mean of their skin spots decreased by 5.5%. Moreover, 9 out of the 10 subjects felt improvement in their skin spots, indicating that the percentage of improved individuals was 90%. From this, it could be known that taking the composition containing the Saskatoon berry extract reduced the skin spots, and effectively helped to fade color spots.

See FIG. 9. One of the subjects had their facial spots photographed at week 0 and week 4. It could be seen that the number of the spots detected by an instrument at week 4 was reduced significantly.

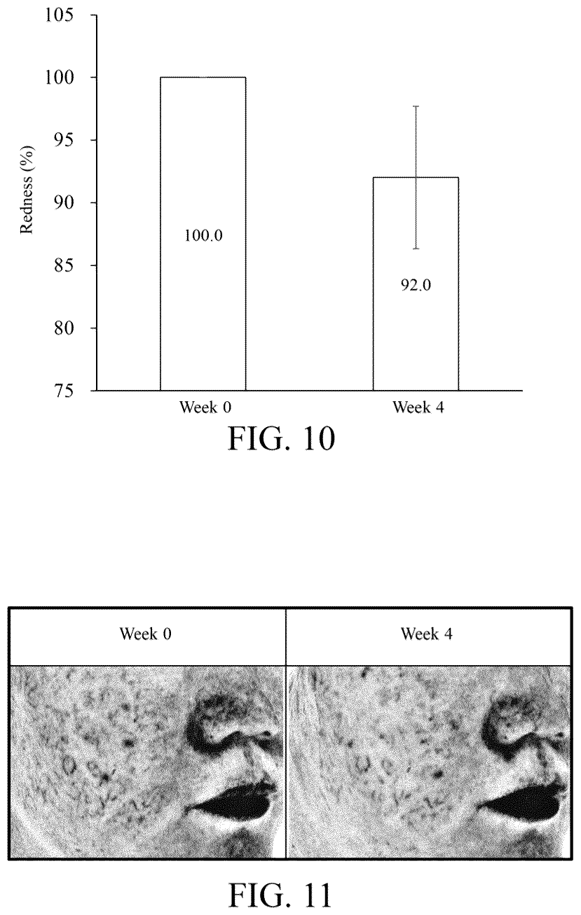

Facial skin was photographed for skin redness using the RBX polarized light technology to detect deep blood vessels or heme of the skin. The higher the measurement value, the more severe the skin redness. See FIG. 10. An average skin redness percentage (%) detected by the VISIA high-quality digital skin analysis system before the 10 subjects took the Saskatoon berry extract (week 0) was regarded as 100%. After taking the Saskatoon berry extract for 4 consecutive weeks, the 10 subjects had their average skin redness percentage decreased to 92%. In other words, after taking the drink containing the Saskatoon berry extract for 4 consecutive weeks, the 10 subjects had their average skin redness percentage decreased by 8%. Moreover, 8 out of the 10 subjects felt improvement in their skin redness, indicating that the percentage of improved individuals was 80%. From this, it could be known that taking the composition containing the Saskatoon berry extract reduced skin redness, and effectively soothed red skin.

See FIG. 11. One of the subjects had his/her skin redness detected at week 0 and week 4. It could be known that red patches detected by the instrument at week 4 were reduced significantly.

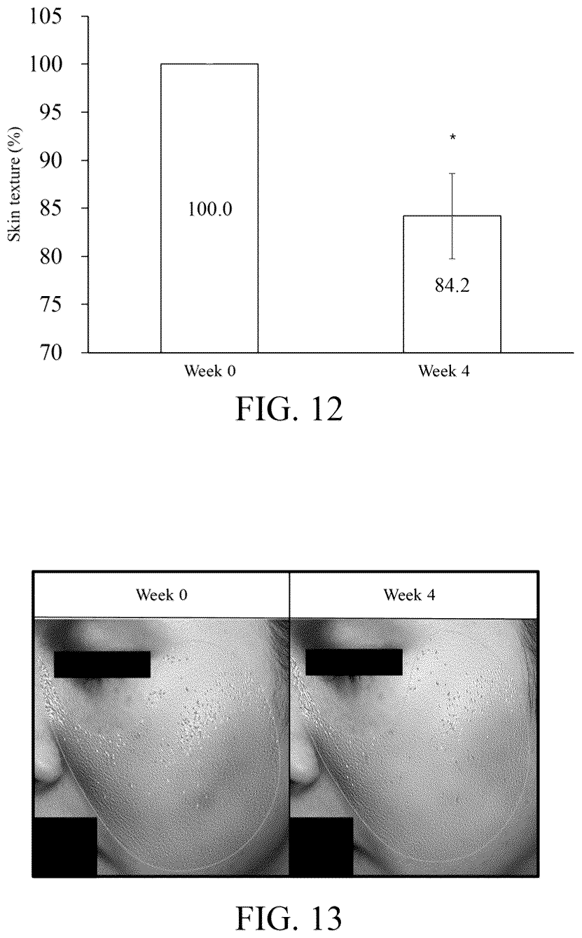

See FIG. 12. A mean of skin texture of the 10 subjects measured at week 0 was considered as 100%. Compared with week 0, a mean of skin texture measured at week 4 was considered as 84.2%. In other words, after taking the drink containing the Saskatoon berry extract for 4 consecutive weeks, the 10 subjects had a mean of their skin texture decreased by 15.8%. Moreover, 9 out of the 10 subjects felt improvement in their skin texture, indicating that the percentage of improved individuals was 90%. From this, it could be known that taking the composition containing the Saskatoon berry extract reduced skin texture, and effectively help smoothened rough texture.

See FIG. 13. One of the subjects had his/her facial texture photographed at week 0 and week 4. It could be seen that the number of texture detected by the instrument at week 4 was reduced significantly.

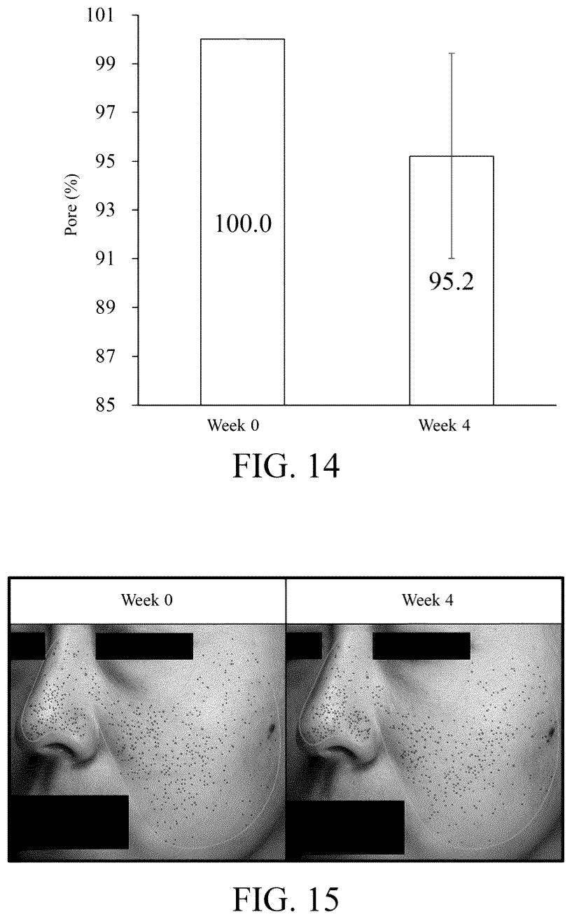

See FIG. 14. A mean of skin pores of the 10 subjects measured at week 0 was considered as 100%. Compared with week 0, a mean of skin pores measured at week 4 was considered as 95.2%. In other words, after taking the drink containing the Saskatoon berry extract for 4 consecutive weeks, the 10 subjects had the mean of their skin pores decreased by 4.8%. Moreover, 7 out of the 10 subjects felt improvement in their skin pores, indicating that the percentage of improved individuals was 70%. From this, it could be known that taking the composition containing the Saskatoon berry extract reduced the number of detected pores, and effectively reduced coarse pores.

See FIG. 15. One of the subjects had his/her facial pores photographed at week 0 and week 4. It could be seen that the number of the pores detected by the instrument at week 4 was reduced significantly.

In summary, according to the use of the Saskatoon berry extract of any embodiment of the present disclosure, the Saskatoon berry extract can be used for preparation of the composition for supplementing iron, promoting iron absorption, and/or improving skin condition. In some embodiments, the Saskatoon berry extract is obtained by extracting the Saskatoon berry with water at 70° C. to 90° C. for 50 min to 70 min. In some embodiments, the Saskatoon berry extract has effects of promoting iron absorption in the intestinal tract, increasing content of iron and ferritin in blood, increasing content of collagen in the skin, increasing secretion of elastin in the skin, and enhancing expression of skin anti-aging genes (such as CCT2 gene, CCT6A gene, NADSYN gene, SOD3 gene, and SIRT1 gene), improving skin conditions (such as reducing skin spots, reducing skin redness, smoothening skin texture, and reducing coarse pores), and the like.

What is claimed is:

1. A method for supplementing iron or promoting iron absorption, comprising administrating to a subject in need thereof a composition comprising a Saskatoon berry extract, wherein the Saskatoon berry extract is obtained by extracting Saskatoon berry (Amelanchier alnifolia) with water.

2. The method according to claim 1, wherein a weight ratio of the water to the Saskatoon berry is 10-20:1.

3. The method according to claim 1, wherein the Saskatoon berry is extracted at 70° C. to 90° C.

4. The method according to claim 1, wherein the Saskatoon berry is extracted for 50 min to 70 min.

5. The method according to claim 1, wherein the Saskatoon berry extract is obtained by extracting the Saskatoon berry with water at 85° C.±5° C. for 60 min, and a weight ratio of the water to the Saskatoon berry is 12:1.

6. The method according to claim 1, wherein the Saskatoon berry extract promotes iron absorption in an intestinal tract of the subject to achieve the effect of promoting iron absorption.

7. The method according to claim 1, wherein the Saskatoon berry extract increases content of iron and ferritin in blood of the subject to achieve the effect of supplementing iron.

8. A method for improving skin condition, comprising administering to a subject in need thereof a composition comprising a Saskatoon berry extract, wherein the Saskatoon berry extract is obtained by extracting Saskatoon berry (Amelanchier alnifolia) with water.

9. The method according to claim 8, wherein a weight ratio of the water to the Saskatoon berry is 10-20:1.

10. The method according to claim 8, wherein the Saskatoon berry is extracted at 70° C. to 90° C.

11. The method according to claim 8, wherein Saskatoon berry is extracted for 50 min to 70 min.

12. The method according to claim 8, wherein the Saskatoon berry extract is obtained by extracting the Saskatoon berry with water at 85° C.±5° C. for 60 min, and a weight ratio of the water and the Saskatoon berry is 12:1.

13. The method according to claim 8, wherein the Saskatoon berry extract increases content of collagen in skin, increases secretion of elastin in skin, or a combination thereof to achieve the effect of improving skin condition.

14. The method according to claim 8, wherein the Saskatoon berry extract enhances expression of anti-aging genes in skin of the subject to achieve the effect of improving skin condition, and the anti-aging genes comprise CCT2 gene, CCT6A gene, NADSYN gene, SOD3 gene, SIRT1 gene, or any combination thereof.

15. The method according to claim 8, wherein the Saskatoon berry extract reduces skin spots, reduces skin redness, smoothens skin texture, and reduces coarse pores, or any combination thereof to achieve the effect of improving skin condition.


SEQUENCE LISTING (10 sequences)

SEQ ID NO: 1 - DNA, 18 bp, synthetic construct
  source 1..18: mol_type = other DNA
aagccacgaaggctgcaa

SEQ ID NO: 2 - DNA, 22 bp, synthetic construct
  source 1..22: mol_type = other DNA
tcatcggaaccatgatcaactg

SEQ ID NO: 3 - DNA, 23 bp, synthetic construct
  source 1..23: mol_type = other DNA
tggccagaacatctcttcgtact

SEQ ID NO: 4 - DNA, 26 bp, synthetic construct
  source 1..26: mol_type = other DNA
agtccactacagcctctgttaagaca

SEQ ID NO: 5 - DNA, 20 bp, synthetic construct
  source 1..20: mol_type = other DNA
gcaaaatgtgcaggctcgaa

SEQ ID NO: 6 - DNA, 20 bp, synthetic construct
  source 1..20: mol_type = other DNA
gcactggagcagtcgtactt

SEQ ID NO: 7 - DNA, 18 bp, synthetic construct
  source 1..18: mol_type = other DNA
agctggaaaggtgcccga

SEQ ID NO: 8 - DNA, 21 bp, synthetic construct
  source 1..21: mol_type = other DNA
cttggcgtacatgtctcggat

SEQ ID NO: 9 - DNA, 23 bp, synthetic construct
  source 1..23: mol_type = other DNA
tagccttgtcagataaggaagga

SEQ ID NO: 10 - DNA, 25 bp, synthetic construct
  source 1..25: mol_type = other DNA
acagcttcttcacagtcaactttgt